(19) United States
(12) Patent Application Publication
(10) Pub. No.: US 2015/0182712 A1
(43) Pub. Date: Jul. 2, 2015

(54) VENTILATOR MANAGEMENT

(71) Applicant: CERNER INNOVATION, INC., Kansas City, KS (US)

(72) Inventors: LISA KELLY, Overland Park, KS (US); JUDITH A. ZAKUTNY, Olathe, KS (US); STEPHANIE L. ROGERS, Kansas City, MO (US)

(73) Assignee: CERNER INNOVATION, INC., Kansas City, KS (US)

(21) Appl. No.: 14/174,576

(22) Filed: Feb. 6, 2014

Related U.S. Application Data
(60) Provisional application No. 61/921,778, filed on Dec. 30, 2013.

(51) Int. Cl. A61M 16/00; A61M 16/04

(52) U.S. Cl. CPC A61M 16/0051; A61M 16/0069; A61M 16/0003; A61M 16/04; A61M 2205/52; A61M 2205/505; A61M 2205/3303; A61M 2230/005; A61M 2230/20; A61M 2230/42; A61M 2230/437; A61M 2016/0027; A61M 2205/18; A61M 2205/3553; A61M 2205/3584

(57) ABSTRACT

Methods, computer systems and computer readable media for communicating data to and receiving data from ventilators in a healthcare setting and displaying the data on a user device are provided. A ventilator order is communicated to a ventilator associated with a patient. A continuous data feed is received from the ventilator. In embodiments, the continuous data feed includes a ventilator rate, a respiratory rate, a positive inspiratory rate, a positive end expiratory rate, and a mean airway pressure. Discrete information associated with the ventilator is received. In embodiments, the discrete information includes endotracheal tube (ETT) size, ETT placement, ETT mode changes, ETT events, and ETT status. Additional information including blood gas results, sedation/paralytic medication information, and calculations, the calculations determined by ventilator data from the continuous data feed and/or electronic medical record (EMR) data received from the EMR is received. Data received from the ventilator is displayed. In embodiments, the data includes the continuous data feed, the discrete information, the additional data, ventilator pressure waveforms, patient-ventilator system check information, and diagnostic images.

### CROSS-REFERENCE TO RELATED APPLICATIONS

This application claims priority to U.S. Provisional Application Ser. No. 61/921,778, filed Dec. 30, 2013, entitled “VENTILATOR MANAGEMENT,” which is incorporated herein by reference in its entirety.

### BACKGROUND

Ventilators move air into and out of the lungs and provide a mechanism of breathing for patients who are physically unable to breathe or unable to breathe sufficiently. Typically, ventilators are utilized by patients in intensive care, emergency medicine, and under anesthesia.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims.

Methods, computer systems and computer readable media for communicating data to and receiving data from ventilators in a healthcare setting and displaying the data on a user device are provided. A ventilator order is communicated to a ventilator associated with a patient. A continuous data feed is received from the ventilator. In embodiments, the continuous data feed includes a ventilator rate, a respiratory rate, a positive inspiratory rate, a positive end expiratory rate, and a mean airway pressure. Discrete information associated with the ventilator is received. In embodiments, the discrete information includes endotracheal tube (ETT) size, ETT placement, ETT mode changes, ETT events, and ETT status. Additional information including blood gas results, sedation/paralytic medication information, and calculations, the calculations determined by ventilator data from the continuous data feed and/or electronic medical record (EMR) data received from the EMR is received. Data received from the ventilator is displayed. In embodiments, the data includes the continuous data feed, the discrete information, the additional data, ventilator pressure waveforms, patient-ventilator system check information, and diagnostic images (e.g., chest x-rays in the context of the ventilator data).

### BRIEF DESCRIPTION OF THE DRAWINGS

Embodiments are described in detail below with reference to the attached drawing figures, wherein:

FIG. 1 is a block diagram of an exemplary computing environment suitable to implement embodiments of the present invention;

FIG. 2 is an exemplary system architecture suitable to implement embodiments of the present;

FIG. 3 is a screenshot of a graphical user interface in accordance with embodiments of the present invention; and

FIG. 4 is a flow diagram of a method in accordance with an embodiment of the present invention.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

Embodiments of the present invention are directed methods, computer systems and computer readable media for receiving data from ventilators in a healthcare setting and displaying the data on a user device. Centralized clinician views are provided to manage individual and multiple patients associated with ventilators. Embodiments provide near real-time graphical displays of ventilator data to clinicians. In addition, near real-time graphical displays of patient physiologic data is displayed simultaneously to a clinician along with the ventilator data. This allows for clinician verification of the ventilator data received to be completed within context of the patient's hemodynamic and vital sign documentation. Embodiments of the present invention enable clinicians to communicate orders to the ventilators, and present and record data relevant to a patient associated with a ventilator and a treatment the patient is receiving. Embodiments of the present invention remove a clinician, such as a respiratory therapist, from being the integrator of ventilators and data. Pro-active ventilator status and alerts are provided in near real-time to clinicians, resulting in increased nursing efficiency.

Accordingly, one embodiment of the present invention is directed to one or more computer storage media having computer-executable instructions embodied thereon, that, when executed by a computing device, cause the computing device to perform a method for displaying ventilator documentation. The method comprises: communicating a ventilator order to a ventilator associated with a patient; receiving a continuous data feed from the ventilator, the continuous data feed including a ventilator rate, a respiratory rate, a positive inspiratory rate, a positive end expiratory rate, and a mean airway pressure; receiving discrete information associated with the ventilator, the discrete information including endotracheal tube (ETT) size, ETT placement, ETT mode changes, ETT events, and ETT status; receiving additional information including blood gas results, sedation/paralytic medication information, and calculations, the calculations determined by ventilator data from the continuous data feed and/or electronic medical record (EMR) data received from the EMR; displaying data received from the ventilator, the data including the continuous data feed, the discrete information, the additional data, ventilator pressure waveforms, patient-ventilator system check information, and diagnostic images.

Another embodiment of the present invention is directed to a system for displaying ventilator documentation. The system includes one or more processors coupled to a computer storage medium, the computer storage medium having stored thereon a plurality of computer software components executable by the processor. The computer software components include: a continuous data feed display area that displays a continuous data feed received a ventilator, the continuous data feed including a ventilator rate, a respiratory rate, a positive inspiratory rate, a positive end expiratory rate, and a mean airway pressure; a discrete information display area that displays discrete information associated with the ventilator, the discrete information including endotracheal tube (ETT) size, ETT placement, ETT mode changes, ETT events, and ETT status; and an additional information display area that displays additional information including blood gas results, sedation/paralytic medication information, and calculations, the calculations determined by ventilator data from the continuous data feed and/or electronic medical record (EMR) data received from the EMR.

Yet another embodiment of the present invention is directed to computer storage media having computer-executable instructions embodied thereon that, when executed by one or more computing devices, cause the one or more computing devices to produce a graphical user interface (GUI) for displaying ventilator documentation. The GUI comprises: The GUI comprises: a ventilator transmitting data to a server comprising at least one component; an order component that communicates a ventilator order to the ventilator associated with a patient; a continuous data feed component that receives a continuous data feed from the ventilator associated with the patient, the continuous data feed including a ventilator rate, a respiratory rate, a positive inspiratory rate, a positive end expiratory rate, and a mean airway pressure; a discrete information component that receives discrete information associated with the ventilator, the discrete information including endotracheal tube (ETT) size, ETT placement, ETT mode changes, ETT events, and ETT status; an additional information component that receives additional information including blood gas results, sedation/paralytic medication information, and calculations, the calculations determined by ventilator data from the continuous data feed and/or electronic medical record (EMR) data received from the EMR; and a display component that enables data received from the ventilator to be displayed on a clinical user device, the data including the continuous data feed, the discrete information, the additional data, and ventilator pressure waveforms.

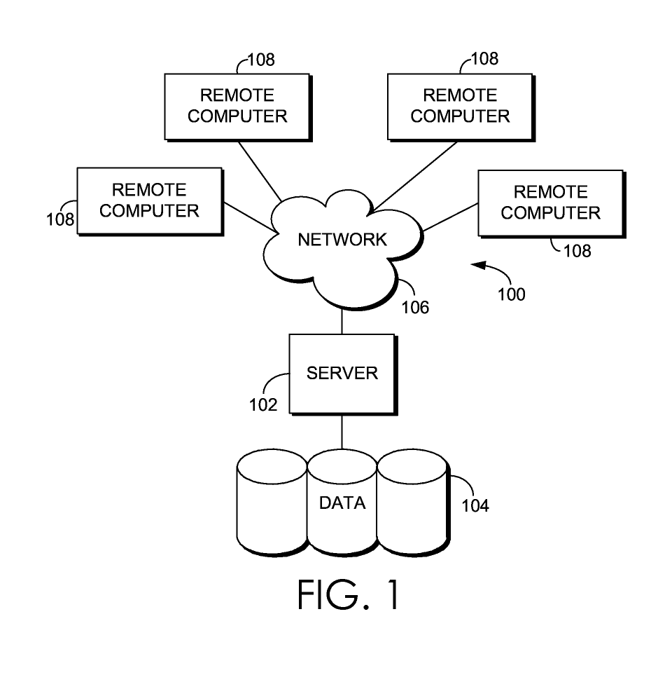

Having briefly described embodiments of the present invention, an exemplary operating environment suitable for use in implementing embodiments of the present invention is described below. Referring to FIG. 1 an exemplary computing environment (e.g., medical-information computing-system environment) with which embodiments of the present invention may be implemented is illustrated and designated generally as reference numeral 100. The computing environment 100 is merely an example of one suitable computing environment and is not intended to suggest any limitation as to the scope of use or functionality of the invention. Neither should the computing environment 100 be interpreted as having any dependency or requirement relating to any single component or combination of components illustrated therein.

The present invention might be operational with numerous other general purpose or special purpose computing system environments or configurations. Examples of well-known computing systems, environments, and/or configurations that might be suitable for use with the present invention include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention might be described in the general context of computer-executable instructions, such as program modules, being executed by a computer. Exemplary program modules include routines, programs, objects, components, and data structures that perform particular tasks or implement particular abstract data types. The present invention might be practiced in distributed computing environments where tasks are performed by remote processing devices that are linked through a communications network. In a distributed computing environment, program modules might be located in association with local and/or remote computer storage media (e.g., memory storage devices).

With continued reference to FIG. 1, the computing environment 100 includes a general purpose computing device in the form of a control server 102. Exemplary components of the control server 102 include a processing unit, internal system memory, and a suitable system bus for coupling various system components, including database cluster 104, with the control server 102. The system bus might be any of several types of bus structures, including a memory bus or memory controller, a peripheral bus, and a local bus, using any of a variety of bus architectures. Exemplary architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 102 typically includes therein, or has access to, a variety of computer-readable media, for instance, database cluster 104. Computer-readable media can be any available media that might be accessed by server 102, and includes volatile and nonvolatile media, as well as, removable and nonremovable media. Computer-readable media might include computer storage media. Computer storage media includes volatile and nonvolatile media, as well as, removable and nonremovable media implemented in any method or technology for storage of information, such as computer-readable instructions, data structures, program modules, or other data. In this regard, computer storage media might include RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the control server 102. Combinations of any of the above also may be included within the scope of computer-readable media.

The computer storage media discussed above and illustrated in FIG. 1, including database cluster 104, provide storage of computer-readable instructions, data structures, program modules, and other data for the control server 102.

The control server 102 might operate in a computer network 106 using logical connections to one or more remote computers 108. Remote computers 108 might be located at a variety of locations in a medical or research environment, including clinical laboratories (e.g., molecular diagnostic laboratories), hospitals and other inpatient settings, ambulatory settings, medical billing and financial offices, hospital administration settings, home healthcare environments, and clinicians' offices. Clinicians might include a treating physician or physicians; specialists such as surgeons, radiologists, cardiologists, and oncologists; emergency medical technicians; physicians' assistants; nurse practitioners; nurses; nurses' aides; pharmacists; dieticians; microbiologists; laboratory experts; laboratory technologists; genetic counselors; researchers; respiratory therapists; students; and the like. The remote computers 108 might also be physically located in nontraditional medical care environments so that the entire healthcare community might be capable of integration on the network. The remote computers 108 might be personal computers, servers, routers, network PCs, peer devices, other common network nodes, or the like and might include some or all of the elements described above in relation to the control server 102. The devices can be personal digital assistants or other like devices.

Exemplary computer networks 106 include local area networks (LANs) and/or wide area networks (WANs). Such networking environments are commonplace in offices, enterprise-wide computer networks, intranets, and the Internet. When utilized in a WAN networking environment, the control server 102 might include a modem or other means for establishing communications over the WAN, such as the Internet. In a networked environment, program modules or portions thereof might be stored in association with the control server 102, the database cluster 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) might be utilized.

In operation, a clinician might enter commands and information into the control server 102 or convey the commands and information to the control server 102 via one or more of the remote computers 108 through input devices, such as a keyboard, a pointing device (commonly referred to as a mouse), a trackball, or a touch pad. Other input devices include microphones, satellite dishes, scanners, or the like. Commands and information might also be sent directly from a remote healthcare device to the control server 102. In addition to a monitor, the control server 102 and/or remote computers 108 might include other peripheral output devices, such as speakers and a printer.

Although many other internal components of the control server 102 and the remote computers 108 are not shown, those of ordinary skill in the art will appreciate that such components and their interconnection are well known. Accordingly, additional details concerning the internal construction of the control server 102 and the remote computers 108 are not further disclosed herein.

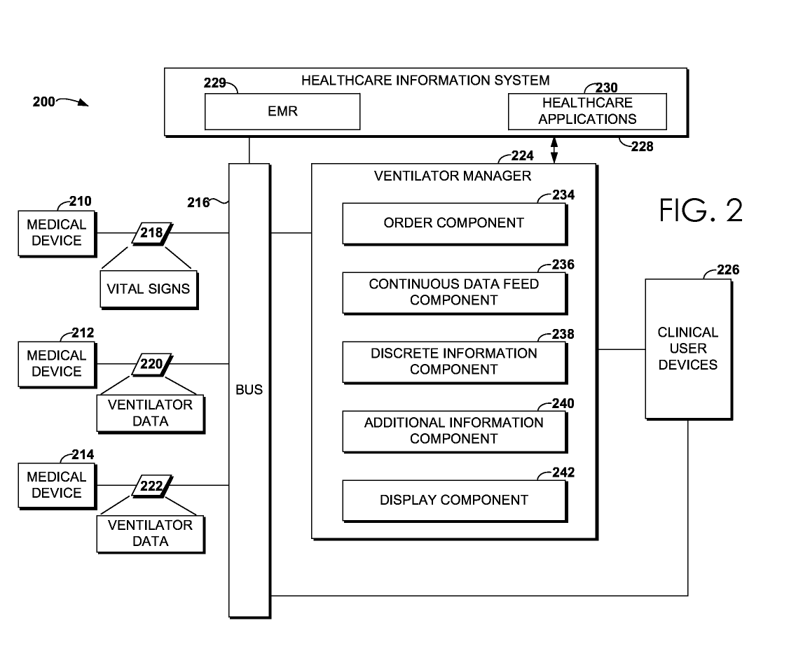

Turning now to FIG. 2, a schematic diagram depicts an operating environment, identified generally by reference numeral 200, suitable to practice an embodiment of the present invention. FIG. 2 includes various components that communicate with one another, including medical device 210, ventilators 212, 214, clinical user device 226, bus 216, ventilator manager 224, and healthcare information system 228. In one embodiment of the present invention, data generated by a medical device 210 or a ventilator 212, 214 is routed to and managed by ventilator manager 224, as opposed to, each medical device 210 and ventilator 212, 214 displaying information on the medical device or ventilator respectively. For example, data 218, 220, 222 is communicated to bus 216, which might then forward the data to ventilator manager 224 to be further processed and routed. Before describing in more detail how these components communicate, each component will be generally described.

In an embodiment of the present invention, medical device 210 might include cardiac monitors, infusion pumps, balloon pumps, patient beds, sequential-compression devices, electronic security devices, and vital-sign detecting devices. Medical device 210 may generate various data (e.g., measured heart rate) that, as described in more detail below, is communicated to other components (e.g., bus 216) of operating environment 200. Moreover, medical device 210 might also receive information from components of operating environment 200.

Ventilators 212, 214 generate various data, including, but not limited to, a ventilator rate, a respiratory rate, a positive inspiratory rate, a positive end expiratory rate, and a mean airway pressure. Discrete information associated with ventilators 212, 214 may include ETT size, ETT placement, ETT mode changes, ETT events, and ETT status. This data is communicated to other components (e.g., bus 216) of operating environment 200. Moreover, ventilators 212 and 214 might also receive information from components of operating environment 200.

Healthcare information system 228 includes an integrated system of healthcare-related information that is usable by a healthcare facility to operate and provide patient care. For example, healthcare information system 228 includes an electronic medical record 229 (also referred to herein as “EMR”) and a healthcare applications component 230. EMR 229 includes an electronic version of patient records including information for the patient, such as ventilator data, medication and infusion orders, tasks, images, examination reports, testing and lab results, medical history, etc. Healthcare applications component 230 includes information that is input and provided at a patient's point-of-care (e.g., patient bedside) to assist healthcare professionals to provide appropriate care. An exemplary applications component 230 includes a patient order entry component for entering electronic healthcare orders for a patient. In an embodiment of the present invention, healthcare information system 228 receives information from other components, as will be described in more detail below. Moreover, healthcare information system 228 might also provide information that is communicated to other components of operating environment 200.

Clinical user devices 226 include devices that are used within a healthcare facility to receive, display, and send information to a user, such as a clinician. Clinical user devices 226 also facilitate requests to receive additional information. Exemplary clinical user devices 226 include personal communication devices, a clinician computer workstation, and an email system. Personal communication devices include devices that are used by an individual to receive and send information, such as an in-house phone, a pager, and a mobile device. Workstations include a remote computer terminal that is used to present information to a user, such as a clinician, and receive input. Workstations might be set up at a nurse's station to or at a patient bedside. Accordingly, in an embodiment of the present invention, clinical user devices 226 present to users information that is received from other components of operating environment 200. Moreover, clinical user devices 226 might also receive inputs from a clinician that are communicated to other components of operating environment 200. Clinical user devices 226 also communicate to other components of operating environment 200 requests to receive additional information. For example, clinical user devices 226 might communicate information to ventilator manager 224, HIS 228, EMR 229, and medical devices 210, 212 and 214.

As previously indicated, and as depicted in FIG. 2, each of medical devices 210, ventilators 212 and 214, healthcare information system 228, and clinical user devices 226 may be in communication with bus 216. Bus 216 generally provides a connection framework for these components by creating and managing all connections, providing a messaging architecture to facilitate an exchange of information between the various components of FIG. 2, and providing general operational and management capabilities for connected devices. In one embodiment, medical device 210, ventilators 212 and 214, clinical user devices 226, and healthcare information system 228 communicate with bus 216 as described in U.S. patent application Ser. No. 12/347,475 (U.S. Pat. App. '475), which is incorporated herein by reference. For example, ventilators 212 and 214 might include various different types of ventilators that are manufactured by various different vendors. As such, components of FIG. 2 might communicate with bus 216 via a gateway (e.g., device gateway or internal gateway), an adapter, or by any other means described by U.S. Pat. App. '475. In a further embodiment, bus 216 includes those capabilities described in U.S. Pat. App. '475. As indicated in U.S. Pat. App. '475, once data is received (e.g., data 218, 220, 222, and 227) it can be sorted and routed to other applications.

In an embodiment of the present invention, such applications are included in a ventilator manager 224. As such, bus 216 might receive information (e.g., data 218, 220, 222) and route the data to ventilator manager 224. Moreover, bus 216 might receive information from clinical user devices 226 and route the information to ventilator manager 224. In a further embodiment, bus 216 receives information from healthcare information system 228 and routes the information to ventilator manager 224. In another embodiment, bus 216 receives information from ventilator manager 224 and routes the information to other components. For example, bus 216 routes information from clinical user devices 226 to healthcare information system 228.

In an embodiment of the present invention, ventilator manager 224 communicates with bus 216 and functions to consolidate and manage information received from the various components of operating environment 200. In this embodiment, instead of components communicating directly with one another, information is routed through and processed by ventilator manager 224. Ventilator manager 224 allows for consolidation and communication of information from various sources, which may not easily integrated or combinable by direct communication. For example, ventilator manager 224 allows for information from ventilators 212 and 214 to be packaged with information from medical device 210 and healthcare information system 228 in order to generate and communicate a more information-rich notification to a notification recipient (e.g., clinical user device 226). Moreover, a set of normalized information is more easily sorted and reported than a set of information that organized in alternative formats of various information sources. Alternatively, medical device 210, ventilators 212 and 214, clinician user devices 226 and healthcare information system 228 may communicate directly with ventilator manager via a network environment.

Ventilator manager 224 communicates with bus 216 and functions to document, display and manage ventilator information received from ventilators 212 and 214. Ventilator manager 224 includes, in various embodiments, order component 234, continuous data feed component 236, discrete information component 238, additional information component 240, and display component 242. While these components are included in the embodiment of FIG. 2, any number of components, either more or less than the illustrated components, may be used to accomplish the purposes of the present invention. Other components and subcomponents are contemplated to be within the scope of the present invention. Furthermore, although depicted as residing on one device, such as a server, it will be appreciated that any number of components and/or subcomponents may reside on any number of computing devices or servers.

Order component 234 communicates a ventilator order to a ventilator associated with a patient. The ventilator order may also be associated with a particular order associated with the patient that may have particular requirements. This provides a user, such as a clinician, with the opportunity to display and save ventilator and other data to a patient's EMR at an interval, in one embodiment, relevant to a treatment the patient is receiving. For example, a post-operative cardiothoracic surgery order may require monitoring vital signs at specific intervals, providing medications at varying doses through various methods of delivery, such as through an infusion pump, performing laboratory tests at specific times, or performing other actions or activities that are time dependent. A ventilator with particular parameters (e.g., size, placement, and the like) may also be required. Orders for post cardiac catheterization or post carotid arterial stent may have entirely different requirements. To illustrate, an abbreviated sample of two real-world orders are contrasted below to distinguish the requirements for vital signs:

Post-Operative Cardiothoracic Surgery Order

Vital Signs:

Record the following on arrival to ICU: MAP q 5 min×15 min, q 15 min until stable×2 hours, q 30 min×2 hours, q 1 hour, then decrease to q 2 hours when patient stable and prn:
DO NOT WEDGE SWAN
Arterial pressure (from arterial line, record cuff pressure on admission and q shift)
Heart Rate
Respiratory Rate (spontaneous/mechanical)
MAP CVP (direct monitoring)
PA systolic/diastolic pressures SaO₂

Record the temperature on admission and then q 4 hours. Call physician for temperature >38.5 Celsius. Urine culture if indicated. Sputum culture and blood culture×2 q 30 minutes if temperature >38.5 Celsius.

Record urine volume every hour

Record cardiac output/index/SVR on admission, at 1 hour and every 4 hours postoperative (unless weaning vasoactive medication—then 30 minutes to 1 hour after change).

Call physician for:
MAP <60 mmHg or >100 mmHg
Low BP (MAP <60 or SBP <90)
CVP >18 mmHg
Heart rate <60 or >120 or rhythm change
SaO₂<95% (African-American) or <92% (Other)
Cardiac index <2.1 L/min/m₂
Urine output <0.5 mL/kg/hr
Chest tube drainage ≧150 ml/hr

Post Cardiac Catheterization Order

Vital Signs:

Record the following: q 15 min×4, then q 30 min×4 then q 1 hr×4 then routine and prn

For Femoral Sites:

Examine procedural site for swelling/bleeding

Assess distal extremity for warmth, color, sensation, and presence of pulse with each vital sign

NOTIFY cardiologist for uncontrollable bleeding, hematoma, loss of pulse in affected extremity, SBP <80 mm or HR >110

For Brachial/Radial Caths:

Check right brachial/radial area for bleeding and radial pulse every 30 min×6

Keep arm straight for 1 hour

Check hand for pain, numbness, and capillary refill with each vital sign.

Orders for various treatments often span many pages and include items such as vital signs, general measures, extubation, medications, laboratory testing, among many others. In one embodiment, a protocol corresponds to the unique requirements evidenced by the order for a specific treatment. For example, a clinician may select, in one embodiment, a protocol corresponding to a post-operative cardiothoracic surgery order. In another embodiment, a clinician may select a protocol corresponding to a post carotid arterial stent order. In yet another embodiment, a clinician may select a protocol corresponding to a conscious sedation procedure. A clinician may select, in one embodiment, a protocol corresponding to a rapid sequence intubation procedure. In another embodiment, a clinician may select a protocol corresponding to a pacemaker insertion procedure. A clinician may select, in one embodiment, an acute myocardial infarction protocol. In another embodiment, a clinician may select a stroke protocol.

The order component 234 may further associate the ventilator for a patient and an order for the patient in response to receiving an indication that ventilator and patient order are to be associated. In one embodiment, identifications of the patient and ventilator are received. The identifications may be received in a number of ways, including, but not limited to, scanning a barcode associated with the patient and/or ventilator, entering a name or identification associated with the patient and/or ventilator, or searching an electronically searchable database for a patient and/or ventilator.

This indication to associate a ventilator and patient order may take many forms. An order is an instruction or request for a procedure, a medication, a ventilator, infusion fluid, nutrients, a laboratory test, an evaluation, a treatment, or a nursing task to be performed for a patient. An explicit association may be available to the user, such as through a selectable button on a graphical user interface displayed on the clinical user device 226. The patient order and ventilator 212, 214 may be associated prior to, simultaneously with or after receiving data from a ventilator 212, 214. Order component 234 may suggest orders to associate with a ventilator 212, 214. For example, order component 234 may filter patient orders to display only orders to be administered by ventilator 212, 214.

Order component 234 may further present the clinician with an interface that, in one embodiment, comprises a drop down box, allowing the clinician to select, from a list of available protocols, the desired protocol. In another embodiment, the protocols appear in order of most frequent use.

By selecting an order via the order component 234, the order component 234 communicates with the medical device 210 and ventilator 212, 214, as necessary, such that the appropriate data is communicated and displayed and if desired, transmitted to the patient's EMR. This avoids the onerous and time-consuming task of requiring a clinician to manually collect and record data, or manually alter collection and recordation times. Accordingly, the clinician is able to spend more time caring for the patient and less time reviewing and fulfilling requirements associated with each individual patient's order.

Continuous data feed component 236, discrete information component 238, and additional information component 240 acquire or receive data from a ventilator 212, 214 or medical device 210 that has been associated with a patient and/or order for the patient. The type of data that may be received information regarding a ventilator rate, a respiratory rate, a positive inspiratory rate, a positive end expiratory rate, a mean airway pressure, ETT size, ETT placement, ETT mode changes, ETT events, and ETT status, blood gas results, sedation/paralytic medication information, calculations, and alerts. Continuous data feed component 236, discrete information component 238, and additional information component 240 may also receive data from medical devices other than ventilators, such as vital sign and blood pressure monitors. The data is in computerized form and is communicated in electronic form to the BUS and/or event manager without any user intervention. For example, the device would automatically be sent to the BUS and/or infusion manager without a user, such as a nurse or clinician, having to manually key-in or enter any information into a computer system.

Continuous data feed component 236, discrete information component 238, and additional information component 240 continually receive data from the associated ventilators and medical devices as long as they are associated to the patient and/or patient's order. A continuous feed of data may be fed from the ventilator and/or medical device to bus 216 and then to ventilator manager 224. Continuous data feed component 236 receives a continuous data feed from the ventilator. The continuous data feed may include a ventilator rate, a respiratory rate, a positive inspiratory rate, a positive end expiratory rate, and a mean airway pressure. Discrete information component 238 receives discrete information associated with the ventilator. The discrete information may include ETT size, ETT placement, ETT mode changes, ETT events, and ETT status. Additional information component 240 receives additional information including blood gas results, sedation/paralytic medication information, and calculations. The calculations may be determined by ventilator data from the continuous data feed and/or electronic medical record (EMR) data received from the EMR. In one embodiment, the data received from the ventilators and medical devices can be manipulated (e.g., by a clinician) prior to being stored in the patient's EMR. The data may be stored in an application or service such that a user can edit the data prior to the data being transmitted to the patient's EMR.

Continuous data feed component 236, discrete information component 238, and additional information component 240 may further determine the status of the device based on data received from a ventilator. The status may include whether or not a device is connected to the system or if it has lost connectivity, whether a ventilator is operating or has been stopped. In one embodiment, if the ventilator manager 224 does not receive any data from a ventilator (e.g., such as a heartbeat signal of the device or any other data) it will be determined that the ventilator has lost connectivity.

In another embodiment, ventilator manager 224 may not receive any information about rate or pressure but still receives an indication given at a certain interval of time that a particular ventilator is connected to bus 216. Based on this data, continuous data feed component 236, discrete information component 238, or additional information component 240 determines that the ventilator has been stopped but the ventilator is still connected. Continuous data feed component 234, discrete information component 238, and additional information component 240, if needed, also performs any necessary conversions on the data received from the ventilator needed to determine the associated rates, pressures, and calculations or type of alert needed. In addition, continuous data feed component 234, discrete information component 238, and additional information component 240 may rate alert information received from the ventilator by level of severity. The level of severity may be represented by an icon displayed for the alert by display component 242.

Continuous data feed component 236 may also generate an alert that data received from a ventilator does not match the associated order. For example, if the rates and/or pressure received from the data from the ventilator do not match the rate of the associated order, continuous data feed component 236 generates an alert notifying a clinician of the discrepancy. In addition, continuous data feed component 236 can access current electronic orders to be administered by ventilator for the patient and suggest a more recent version of an order or the closest order that may fit the data being received by the ventilator.

Display component 242 enables data received from the ventilator to be displayed on a clinical user device 226. The data may include the continuous data feed, the discrete information, the additional data, and ventilator pressure waveforms. The clinical user devices 226 are separate devices from the medical device 210 and ventilators 212 and 214. Display component 242 can display a variety of information received from a ventilator in a variety of formats. For example, the data may be displayed pertaining to the most recent twenty-four hour period and may allow the clinician to zoom in as well as scroll back over a previous time period (e.g., the last seventy-two hours). The data may be presented in a unit device view, displaying all devices currently associated and may allow a clinician to view alerts and/or real-time data associated with each device. A clinician may filter the data associated with the unit device view to view a display of real-time ventilator information for each patient in the unit device view. Similarly, the clinician may view a more detailed view for each patient that may include additional information, such as information extracted or included in an EMR associated with the patient.

Alerts from the data received from the ventilator may be displayed along with textual icon and/or color coding indicating the severity of the alert. For example, a clinician may be alerted that the order associated with ventilator does not match the data being received from the ventilator may also be displayed. Clinical user device 226 may display ventilator data for individual patients or for multiple patients simultaneously.

Display component 242 also provides a user, such as a clinician, with the opportunity to review the data acquired from the ventilator. The data acquired from the ventilator and vital signs collected by other medical devices are displayed to the user in a format that allows the user to edit the data received from the ventilator in context of the patient's vital signs, if desired. Alternatively, the user may authenticate the data as received from the medical device. Once the data received from the ventilator has been reviewed by a clinician, and once the user has had the opportunity to edit or add any other information, the user may select a button, such as a sign button, that indicates that the data is ready to be transmitted or published to the patient's EMR.

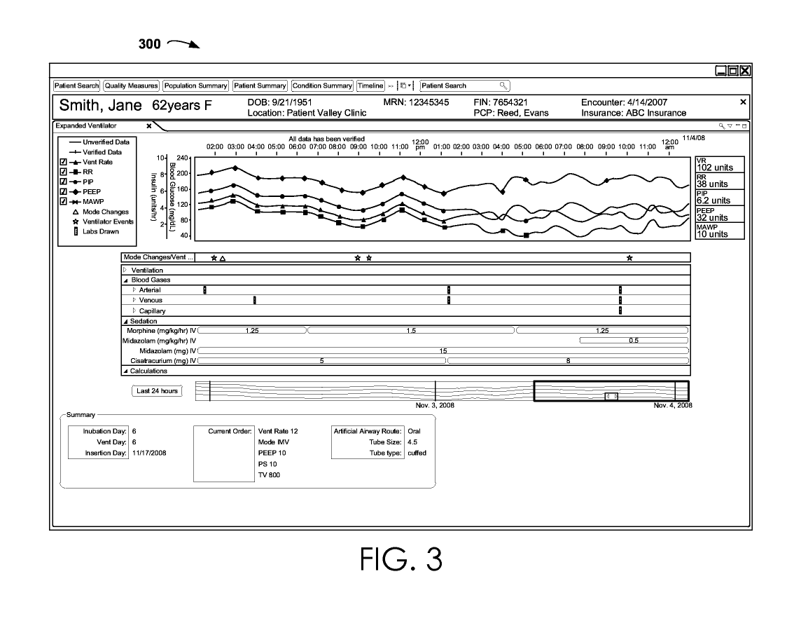

In FIG. 3, an illustrative screen display 300 of an embodiment of the present invention is shown. A continuous data feed display area displays a continuous data feed received from ventilator. The continuous data feed may include a ventilator rate, a respiratory rate, a positive inspiratory rate, a positive end expiratory rate, and a mean airway pressure. The continuous data feed display area may further distinguish between verified and unverified data. The continuous data feed display area may further indicate points in time where a mode changed, an event occurred, or labs were drawn. In addition to displaying real-time ventilator data in graph form, current real-time information may also be displayed in textual format at the current point in time the display is being viewed. A discrete information display area displays discrete information associated with the ventilator. The discrete information may include ETT size, ETT placement, ETT mode changes, ETT events, and ETT status. The discrete information may also include the current day for intubation or on a ventilator and an insertion date. An additional information display area displays additional information including blood gas results, sedation/paralytic medication information, and calculations. The calculations may be determined by ventilator data from the continuous data feed and/or electronic medical record (EMR) data received from the EMR.

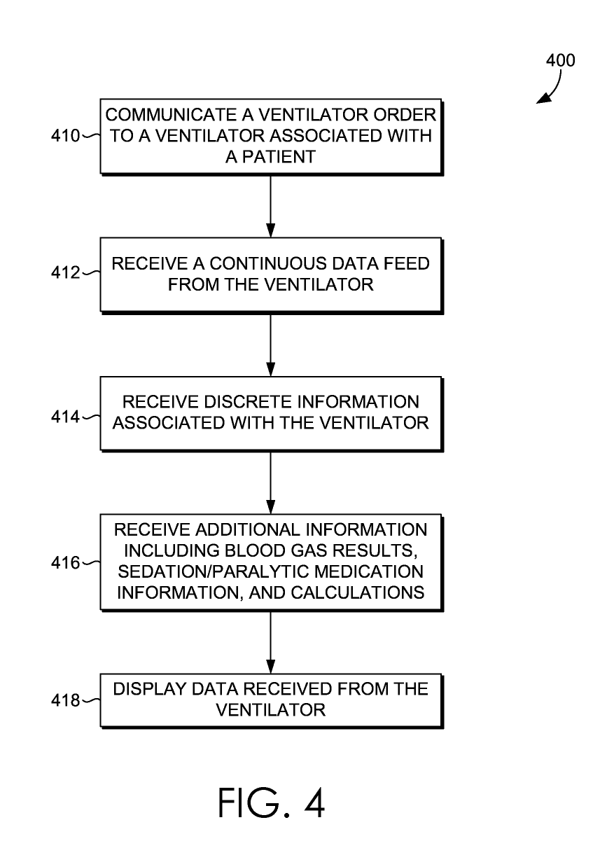

Turning now to FIG. 4, an illustrative flow diagram 400 is shown of a method for displaying ventilator documentation, in accordance with an embodiment of the present invention. At step 410, a ventilator order is communicated to a ventilator associated with a patient. Each ventilator order may be unique based on a condition or treatment of the patient associated with the particular ventilator. At step 412, a continuous data feed is received from the ventilator. The continuous data feed may include a ventilator rate, a respiratory rate, a positive inspiratory rate, a positive end expiratory rate, and a mean airway pressure. At step 414, discrete information associated with the ventilator is received. The discrete information may include endotracheal tube (ETT) size, ETT placement, ETT mode changes, ETT events, and ETT status. At step 416, additional information including blood gas results, sedation/paralytic medication information, and calculations is received. The calculations may be determined by ventilator data from the continuous data feed and/or electronic medical record (EMR) data received from the EMR. At step 418, data received from the ventilator is displayed. The data may include the continuous data feed, the discrete information, the additional data, and ventilator pressure waveforms.

Many different arrangements of the various components depicted, as well as components not shown, are possible without departing from the scope of the claims below. Embodiments of our technology have been described with the intent to be illustrative rather than restrictive. Alternative embodiments will become apparent to readers of this disclosure after and because of reading it. Alternative means of implementing the aforementioned can be completed without departing from the scope of the claims below. Certain features and subcombinations are of utility and may be employed without reference to other features and subcombinations and are contemplated within the scope of the claims.

What is claimed is:

1. One or more computer storage media having computer-executable instructions embodied thereon, that when executed by a computing device, cause the computing device to perform a method for displaying ventilator documentation, the method comprising:
communicating a ventilator order to a ventilator associated with a patient;
receiving a continuous data feed from the ventilator;
receiving discrete information associated with the ventilator;
receiving additional information including blood gas results, sedation/paralytic medication information, and calculations;
displaying data received from the ventilator.

2. The media of claim 1, wherein the continuous data feed includes a ventilator rate, a respiratory rate, a positive inspiratory rate, a positive end expiratory rate, and a mean airway pressure.

3. The media of claim 1, wherein the discrete information includes endotracheal tube (ETT) size, ETT placement, ETT mode changes, ETT events, and ETT status.

4. The media of claim 1, wherein the calculations are determined by ventilator data from the continuous data feed and/or electronic medical record (EMR) data received from the EMR.

5. The media of claim 1, wherein the data includes the continuous data feed, the discrete information, the additional data, ventilator pressure waveforms, patient-ventilator system check information, and diagnostic images.

6. Computer storage media having computer-executable instructions embodied thereon that, when executed by one or more computing devices, cause the one or more computing devices to produce a graphical user interface (GUI) for displaying ventilator documentation, the GUI comprising:
a continuous data feed display area that displays a continuous data feed received from a ventilator;
a discrete information display area that displays discrete information associated with the ventilator; and
an additional information display area that displays additional information including blood gas results, sedation/paralytic medication information, and calculations, the calculations determined by ventilator data from the continuous data feed and/or electronic medical record (EMR) data received from the EMR.

7. The GUI of claim 6, wherein the continuous data feed includes a ventilator rate, a respiratory rate, a positive inspiratory rate, a positive end expiratory rate, and a mean airway pressure.

8. The GUI of claim 6, wherein the discrete information includes endotracheal tube (ETT) size, ETT placement, ETT mode changes, ETT events, and ETT status.

9. The GUI of claim 6, wherein the continuous data feed display area distinguishes between verified and unverified data.

10. The GUI of claim 9, wherein the continuous data feed display area indicates points in time where a mode changed, an event occurred, or labs were drawn.

11. The GUI of claim 6, wherein real-time information is displayed in textual format at the current point in time each respective display area is being viewed.

12. A computerized system for displaying ventilator documentation, the system comprising:
a ventilator transmitting data to a server comprising at least one component;
an order component that communicates a ventilator order to the ventilator associated with a patient;
a continuous data feed component that receives a continuous data feed from the ventilator associated with the patient, the continuous data feed including a ventilator rate, a respiratory rate, a positive inspiratory rate, a positive end expiratory rate, and a mean airway pressure;
a discrete information component that receives discrete information associated with the ventilator, the discrete information including endotracheal tube (ETT) size, ETT placement, ETT mode changes, ETT events, and ETT status;
an additional information component that receives additional information including blood gas results, sedation/paralytic medication information, and calculations, the calculations determined by ventilator data from the continuous data feed and/or electronic medical record (EMR) data received from the EMR; and
a display component that enables data received from the ventilator to be displayed on a clinical user device, the data including the continuous data feed, the discrete information, the additional data, ventilator pressure waveforms, patient-ventilator system check information, and diagnostic images.

13. The system of claim 12, wherein the display component further provides a user the opportunity to review, edit, and authenticate the data acquired from the ventilator.

14. The system of claim 13, wherein the display component further provides the user a sign button that indicates the data is ready to be transmitted or published to an electronic medical record.

15. The system of claim 12, wherein the display component further presents the data in a unit device view that displays all devices currently associated and allows a clinician to view alerts and/or real-time data associated with each device.

16. The system of claim 12, wherein the display component further provides a detailed view for each patient that includes information extracted or included in an electronic medical record associated with each patient.

17. The system of claim 12, wherein the display component further displays alerts from the data received from the ventilator along with a textual icon and/or color coding indicating the severity of the alert.

18. The system of claim 17, wherein the continuous data feed component further generates an alert that data received form the ventilator does not match the associated order.

19. The system of claim 12, wherein the continuous data feed component further accesses current electronic orders to be administered by the ventilator for the patient and suggests a more recent version of an order or the closest order that fits the data being received by the ventilator.

20. The system of claim 12, wherein the order component further associates the ventilator for a patient and an order for the patient in response to receiving an indication that ventilator and patient order are to be associated.
